(19) United States
(12) Patent Application Publication
(10) Pub. No.: US 2025/0375600 A1
(43) Pub. Date: Dec. 11, 2025

(54) HINGED ANTISEPTIC DELIVERY DEVICE AND METHOD OF USE

(71) Applicant: PROFESSIONAL DISPOSABLES INTERNATIONAL, INC., Woodcliff Lake, NJ (US)

(72) Inventors: Victor Chan, Landing, NJ (US); Gerard Anthony Cahalan, New Milford, CT (US)

(73) Assignee: PROFESSIONAL DISPOSABLES INTERNATIONAL, INC., Woodcliff Lake, NJ (US)

(21) Appl. No.: 18/878,415

(22) PCT Filed: Jul. 12, 2023
(86) PCT No.: PCT/US2023/027443

§ 371 Date: Dec. 23, 2024

Related U.S. Application Data
(60) Provisional application No. 63/389,608, filed on Jul. 15, 2022.

(51) Int. Cl. A61M 35/00; A61B 90/80

(52) U.S. Cl. CPC A61M 35/006; A61B 90/80

(57) ABSTRACT

Hinged antiseptic delivery devices and methods are disclosed comprising a fluid applicator device. The fluid applicator device has a handle having a first end, a second end, and a length therebetween, wherein two fingers extend from the second end and define an opening between the two fingers. The fluid applicator device further has a base plate rotatably coupled with the handle, the base plate having a top surface and a bottom surface. The top surface includes a first support and a second support disposed thereon, and a rod disposed between the first support and the second support. The fluid applicator device further has a foam applicator coupled to the base plate to absorb and distribute an antiseptic solution, wherein the top surface of the base plate is proximate the handle in a delivery condition and is disposed at an angle to the handle in the use condition.

### CROSS-REFERENCE TO RELATED APPLICATION

This application claims priority to U.S. Provisional Patent Application Ser. No. 63/389,608, filed Jul. 15, 2022, which is hereby incorporated by reference herein in its entirety.

### FIELD

The disclosed subject matter relates to a hinged antiseptic delivery device and method of use.

### BACKGROUND

Generally, devices and methods exist for dispensing and applying a product to a surface. For example, paint can be generally applied to a wall with a paint brush, shoe polish can be applied to a shoe with a cloth, and toothpaste can be applied to a toothbrush for cleaning teeth. Such products and devices can be messy and unfortunately spill when presented as separate units. In the medical field, it is common practice to prepare a patient for surgery by applying a fluid product, such as an antiseptic solution, to a target body portion with a device that must be activated prior to use. One of the many problems associated with some typical conventional product or fluid delivery devices is the arrangement of the handle to the applicator, which may give rise to risks such as the clinician contacting the area of the patient's skin that is being prepared for surgery.

To overcome at least certain problems in this field, fluid delivery devices have been designed so that the handle is angled with respect to the applicator. One drawback of such devices is that they are permanently affixed in that position, which require specialized packaging to accommodate the spacing due to the arrangement. Such devices are not efficiently designed for commercial packing and subsequent transportation, as the angled handle in relation to the applicator occupies more volume than a fluid delivery device that is flat.

Thus, there remains a continued need for an improved hinged antiseptic delivery device. The presently disclosed subject matter satisfies these and other needs.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes a fluid applicator device. The fluid applicator device comprises a handle having a first end, a second end, and a length therebetween, wherein two fingers extend from the second end and define an opening between the two fingers; a base plate rotatably coupled with the handle, the base plate having a top surface and a bottom surface, wherein the top surface includes: a first support and a second support disposed thereon, and a rod disposed between the first support and the second support, wherein the rod is housed within the opening between the two fingers, the second end of the handle is configured to at least partially rotate about the rod and is configured to lock with the rod in a use condition; and a foam applicator coupled to the bottom surface of the base plate configured to absorb and distribute an antiseptic solution, wherein the top surface of the base plate is proximate the handle in a delivery condition and the top surface is disposed at an angle to the handle in the use condition.

In accordance with another aspect of the disclosed subject matter, a method of using a fluid applicator device is provided. The method includes providing a fluid applicator device including: a handle having a first end, a second end, and a length therebetween, wherein two fingers extend from the second end and define an opening between the two fingers; a base plate rotatably coupled with the handle, the base plate having a top surface and a bottom surface, wherein the top surface includes: a first support and a second support disposed thereon, and a rod disposed between the first support and the second support, wherein the rod is housed within the opening between the two fingers, the second end of the handle is configured to at least partially rotate about the rod and is configured to lock with the rod in a use condition; and a foam applicator coupled to the bottom surface of the base plate configured to absorb and distribute an antiseptic solution, wherein the top surface of the base plate is proximate the handle in a delivery condition and the top surface is disposed at an angle to the handle in the use condition; rotating the handle from a delivery condition towards a use condition about the rod; and applying an axial force along the handle towards the base plate to lock the handle with the base plate in the use condition.

In accordance with yet another aspect of the disclosed subject matter, a fluid applicator device is provided, comprising a handle having a first end, a second end, and a length therebetween, wherein two fingers extend from the second end and define an opening between the two fingers, wherein the handle further includes at least a first protrusion at the second end; a base plate rotatably coupled with the handle, wherein the at least first protrusion of the handle extends from a first side of the handle to lock the handle at an angle with the base plate, the base plate having a top surface and a bottom surface, wherein the angle is predetermined and can range up to about 90° in relation to the top surface of the base plate, wherein the top surface includes: a first support and a second support disposed thereon, and a rod disposed between the first support and the second support, wherein the rod is housed within the opening between the two fingers, the second end of the handle is configured to at least partially rotate about the rod and is configured to lock with the rod in a use condition, wherein the rod comprises a first indentation and the handle further comprises a first mating member disposed within the opening between the two fingers, the first mating member is receivable in the first indentation to lock the handle with the base plate; and a foam applicator coupled to the bottom surface of the base plate configured to absorb and distribute an antiseptic solution, wherein the top surface of the base plate is proximate the handle in a delivery condition and the top surface is disposed at an angle to the handle in the use condition.

It is to be understood that both the foregoing general description and the following detailed description and drawings are examples and are provided for purpose of illustration and not intended to limit the scope of the disclosed subject matter in any manner.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

The subject matter of the application will be more readily understood from the following detailed description when read in conjunction with the accompanying drawings, in which:

FIG. 1A is a perspective view of a fluid applicator device, according to the disclosed subject matter.

FIG. 1B is an exploded, perspective view of the fluid applicator device of FIG. 1A, according to the disclosed subject matter.

FIG. 2A is a perspective view of a monolithic foam applicator of the fluid applicator device of FIG. 1A, according to the disclosed subject matter.

FIG. 2B is a perspective view of an embodiment of the foam applicator having two sections, according to the disclosed subject matter.

FIG. 2C is a perspective view of another embodiment of the foam applicator having two sections, according to the disclosed subject matter.

FIG. 2D is a perspective view of an embodiment of the foam applicator having perforations, according to the disclosed subject matter.

FIG. 2E is a perspective view of an embodiment of the foam applicator having a two section laminate, according to the disclosed subject matter.

FIG. 3A is a perspective view of a base plate of the fluid applicator device of FIG. 1A, according to the disclosed subject matter.

FIG. 3B is a side, perspective view of a base plate of the fluid applicator device of FIG. 1A, according to the disclosed subject matter.

FIG. 3C is a top, plan view of a base plate of the fluid applicator device of FIG. 1A, according to the disclosed subject matter.

FIG. 3D is a perspective view of an alternative base plate of the fluid applicator device, according to the disclosed subject matter.

FIG. 4 is a perspective view of a base plate of the fluid applicator device of FIG. 1A, according to the disclosed subject matter.

FIG. 5A is a perspective view of an end of a handle of the fluid applicator device of FIG. 1A, according to the disclosed subject matter.

FIG. 5B is a perspective view of an end of a handle of the fluid applicator device of FIG. 1A, according to the disclosed subject matter.

FIG. 6A is a side view of assembling the fluid applicator device of FIG. 1A, according to the disclosed subject matter.

FIG. 6B is a side view of the fluid applicator device of FIG. 1A in a use condition, according to the disclosed subject matter.

FIG. 7A is a cross-sectional view of assembling the fluid applicator device in FIG. 6A, according to the disclosed subject matter.

FIG. 7B is a cross-sectional view of the fluid applicator device in a use condition in FIG. 6B, according to the disclosed subject matter.

FIG. 8 is a side view of the fluid applicator device of FIG. 1A in a delivery condition, according to the disclosed subject matter.

FIG. 9A is a perspective view of a fluid applicator device o with a living hinge, according to an embodiment of the disclosed subject matter.

FIG. 9B is a perspective view of the fluid applicator device of FIG. 9A with a living hinge in a use condition, according to the disclosed subject matter.

FIG. 10A is a side view of the fluid applicator device of FIG. 1A disposed in a packaging container, according to the disclosed subject matter.

FIG. 10B is a top, plan view of the fluid applicator device of FIG. 10A disposed in a packaging container, according to the disclosed subject matter.

FIG. 11A is perspective view of a fluid applicator device with a living hinge in a delivery condition, according to the disclosed subject matter.

FIG. 11B is perspective view of a fluid applicator device with a living hinge in a use condition, according to the disclosed subject matter.

FIG. 11C is partial phantom side view of the fluid applicator device of FIG. 11B, according to the disclosed subject matter.

### DETAILED DESCRIPTION

Reference will now be made in detail to embodiments of the disclosed subject matter, an example of which is illustrated in the accompanying drawings. The disclosed subject matter will be described in conjunction with the detailed description of the system.

In accordance with the disclosed subject matter, a fluid applicator device is provided. The fluid applicator device comprises a handle having a first end, a second end, and a length therebetween, wherein two fingers extend from the second end and define an opening between the two fingers; a base plate rotatably coupled with the handle, the base plate having a top surface and a bottom surface, wherein the top surface includes: a first support and a second support disposed thereon, and a rod disposed between the first support and the second support, wherein the rod is housed within the opening between the two fingers, the second end of the handle is configured to at least partially rotate about the rod and is configured to lock with the rod in a use condition; and a foam applicator coupled to the bottom surface of the base plate configured to absorb and distribute an antiseptic solution, wherein the top surface of the base plate is proximate the handle in a delivery condition and the top surface is disposed at an angle to the handle in the use condition.

In accordance with the disclosed subject matter, a fluid applicator device is provided according to another embodiment. The fluid applicator device includes a handle having a first end, a second end, and a length therebetween, the handle further comprising a peg disposed between the first end and the second end. A base plate is rotatably coupled with the handle by a living hinge, the base plate having a top surface and a bottom surface, the base plate further comprising defining an aperture configured to mate with the peg. A foam applicator is coupled to the bottom surface of the base plate configured to absorb and distribute an antiseptic solution. The peg is configured to lock the handle with the base plate at an angle in a delivery condition.

A method of using the fluid applicator devices described above is also disclosed. The details of the method of using the device will be described in detail in conjunction with the features of the fluid applicator device.

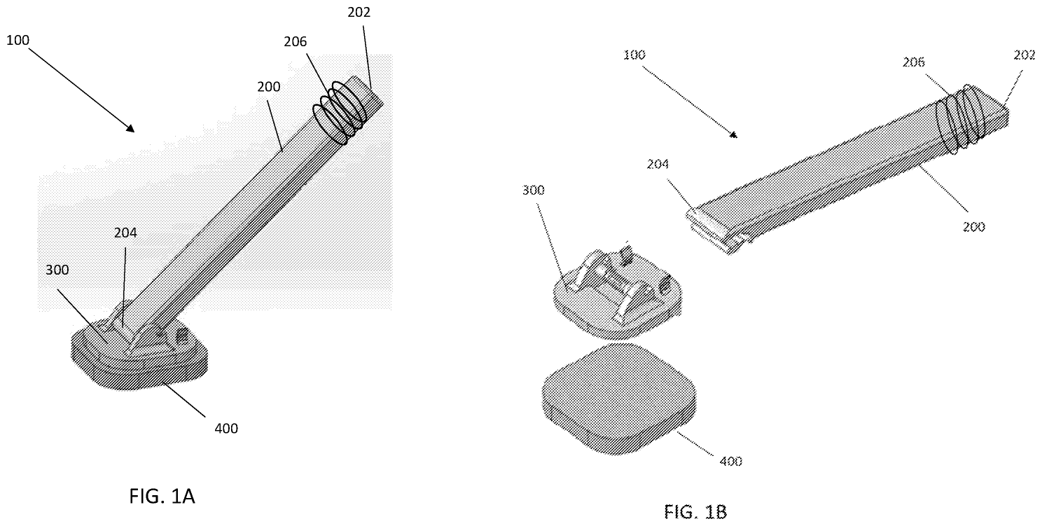

Solely for purpose of illustration, an embodiment of a fluid applicator device 100 is shown in FIGS. 1A-1B. The examples herein are not intended to limit the scope of the disclosed subject matter in any manner. The fluid applicator device 100 can be configured to transition from a delivery condition, while contained with a packaging (not shown), to a use condition for application of a fluid product, such as an antiseptic solution, to a target body portion of a patient's skin. In embodiments, the packaging can be made from any suitable material such as but not limited to a relatively flexible resin material, coated papers, shrink wrap, laminate structures, plastic and metalized films and combinations of thereof, calcium carbonate papers or other synthetic paper including various thermoforming packaging solutions.

As shown in FIGS. 1A-1B, the fluid applicator device 100 can comprise a handle 200, a base plate 300, and a foam applicator 400. The handle 200 can be configured to couple to and rotate about the base plate 300, and the foam applicator 400 can be coupled to a bottom side of the base plate 300 opposite from the handle 200. In embodiments, the delivery condition of the fluid applicator device 100 can be such that the fluid applicator device 100 is generally flat, wherein the length of the handle 200 is substantially parallel to the base plate 300. The use condition of the fluid applicator device 100 can be such that the handle 200 is disposed at an angle to the base plate 300. A user can transition the fluid applicator device 100 from the delivery condition to the use condition once removed from the packaging or once the packaging is at least partially opened. In accordance with these embodiments of the subject matter, the angle of the handle 200 in relation to the base plate 300 in the use condition can be predetermined and can range up to about 90°, and more particularly the handle 200 can be disposed at an angle from about 30° to about 60°.

The handle 200 can be coupled to the base plate 300 through any suitable means, including through the use of fasteners, adhesives, brazing, welding, snap-fit connection, and the like. The handle 200 can have any suitable size and shape. In accordance with these embodiments, the handle 200 can be made out of a plurality of suitable materials. For instance, the handle 200 can be made out of metals, nonmetals, polymers, composites, ceramics, and any combination thereof, and can furthermore be manufactured in a plurality of ways including injection molding. In one embodiment, the handle 200 is made out of polypropylene and can be disposable. In other embodiments, the handle 200 comprises a sterilizable material, such as stainless steel, and can be reusable after cleaning and sterilization. In these embodiments, the foam applicator 400 can be packaged separate from the handle 200, wherein the foam applicator 400 can be coupled to the handle 200 for a single use in a use condition, wherein the handle 200 may be detached from the foam applicator 400 and reusable with a subsequent foam applicator 400. The handle 200 can comprise a first end 202 and a second end 204 opposite to the first end 202, and wherein there is a length therebetween. As illustrated, the second end 204 can couple the handle 200 to the base plate 300. The handle 200 can further comprise grip ribbing 206 at the first end 202 configured to support a hand of a user of the fluid applicator device 100. In other embodiments, the grip ribbing 206 can be disposed along any location of the handle 200 as desired.

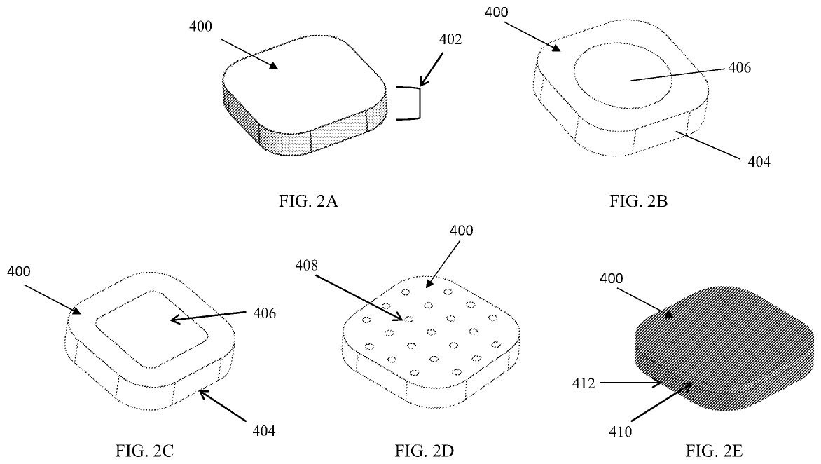

FIGS. 2A-2E depict example embodiments of the foam applicator 400 of the fluid applicator device 100. Examples of suitable foam applicators are described in U.S. Pat. No. 11,160,962, the contents of which is incorporated by reference in its entirety. As embodied herein, the foam applicator 400 can function as a liquid reservoir and can store product such as an antiseptic solution until the delivery device is ready for use.

The foam applicator 400 can function to dispense antiseptic drug product, such as onto a patient's skin. The foam applicator 400 can have any suitable size and shape. The foam applicator 400 is depicted as a square shape with a thickness dimension 402 and rounded corners as an example. In some embodiments, the foam applicator 400 can be of a size such that the foam applicator 400 extends beyond the base plate 300 (referring to FIGS. 1A-1B). The foam applicator 400 can be made of any suitable material for absorbing and dispensing liquids, including but not limited to, reticulated polyester foam, polyurethane foam, a polyester polyurethane blended foam, a polyether polyurethane blended foam, densified foams, felted and non-felted foams, zapped and un-zapped foams, clickable foams, laminated foams, reticulated foams, and the like. Methods of manufacturing foam with various shapes, thicknesses, densities, and pore sizes are well known in the art. For purposes of example, and not limitation, an example of a suitable foam is a polyurethane reticulated foam from manufacture FXI (Media, PA), having a range in porosity dimension from approximately 80 pores per linear inch to approximately 110 pores per linear inch in a disclosed embodiment.

The material, dimensions, density and pore size of the foam applicator 400 can be selected such that the foam applicator 400 can store a desired amount of liquid antiseptic. In one embodiment, the material, dimensions, density and pore size of the foam applicator 400 can be selected such that the foam applicator 400 can store at least 26 ml of antiseptic solution.

The material, dimensions, density and pore size of the foam applicator 400 can further be selected such that liquid antiseptic does not leak or drip from the foam applicator after the foam applicator 400 is removed from a packaging and prior to patient exposure. In some embodiments, and as depicted in FIG. 2A, the foam applicator 400 can have a monolithic foam structure of consistent density, thickness and pore size. In other embodiments, the foam applicator 400 can consist of two different foam structures that can be passively combined, such as but not limited to an interference fit between the foam structures or can be laminated together. For example and not limitation, the foam applicator 400 can have a square outer foam structure 404 with a circular inner foam structure 406, as shown in FIG. 2B. Alternatively, the foam applicator 400 can have a square outer foam structure 404 with a square inner foam structure 406, as shown in FIG. 2C. However, any desired shapes of the inner foam structure and outer foam structure are contemplated herewith. The foam structures of the foam applicator 400 can be selected to prohibit liquid antiseptic from dripping from the foam applicator 400 after the foam applicator 400 is removed from a packaging and prior to patient exposure. For example and not limitation, the outer foam structure 404 can be selected with a density, thickness and pore size configured to provide superior drip prevention, and inner foam structure 406 can be selected with a different density, thickness and pore size configured to facilitate more ready dispensing of the antiseptic to a patient's skin.

In the embodiment of FIG. 2D, the foam applicator 400 further includes at least one through hole 408, as depicted. The through hole(s) 408 can provide additional channels for the antiseptic solution to travel and release onto the patient's skin. As depicted in FIG. 2E, other embodiments of the foam applicator 400 can have two or more foam structures that can be disposed to create a single foam element with a plurality of layers. The foam layers can be layered using any known methods, including but not limited to, flame lamination, ultrasonic welding, and the like. Foam structures can be selected with different properties including but not limited to, density, thickness and pore size to facilitate fluid retention, superior drip prevention and more ready dispensing of the antiseptic to a patient's skin. For example and not for limitation, the upper foam structure 410 can be selected with a density, thickness and pore size configured to provide superior fluid retention and drip prevention, and lower foam structure 412 can be selected with a different density, thickness and pore size configured to facilitate more ready dispensing of the antiseptic to a patient's skin.

As noted above, the product contained by the foam applicator 400 can be suitable for any medical application. For instance, the product can be an antiseptic solution, and application of the solution to a portion of a body can kill microorganisms. In one embodiment, application of the antiseptic solution can kill microorganisms immediately and within approximately 10 minutes and further have a persistent effect for at least 7 hours. As such, the antiseptic solution can be used in preparing the body for surgery. In some embodiments, the antiseptic solution can comprise at least one of chlorhexidine gluconate (CHG), isopropyl alcohol, PVP-I, and PVP-IPA, and mixtures thereof including water. In another embodiment, the antiseptic solution can comprise at least 3.15% w/v chlorhexidine gluconate and 70% v/v isopropyl alcohol (both ±10% w/v of nominal value). The CHG can be designated as: 1,1′-hexamethylenebis[5-(p-chlorophenyl)biguanide] digluconate.

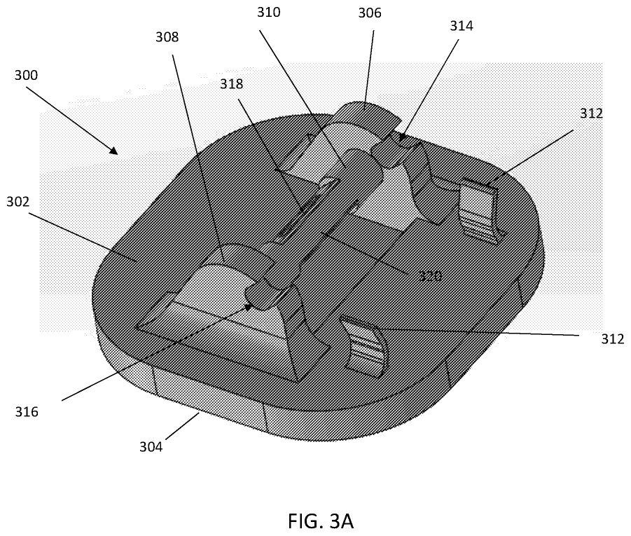

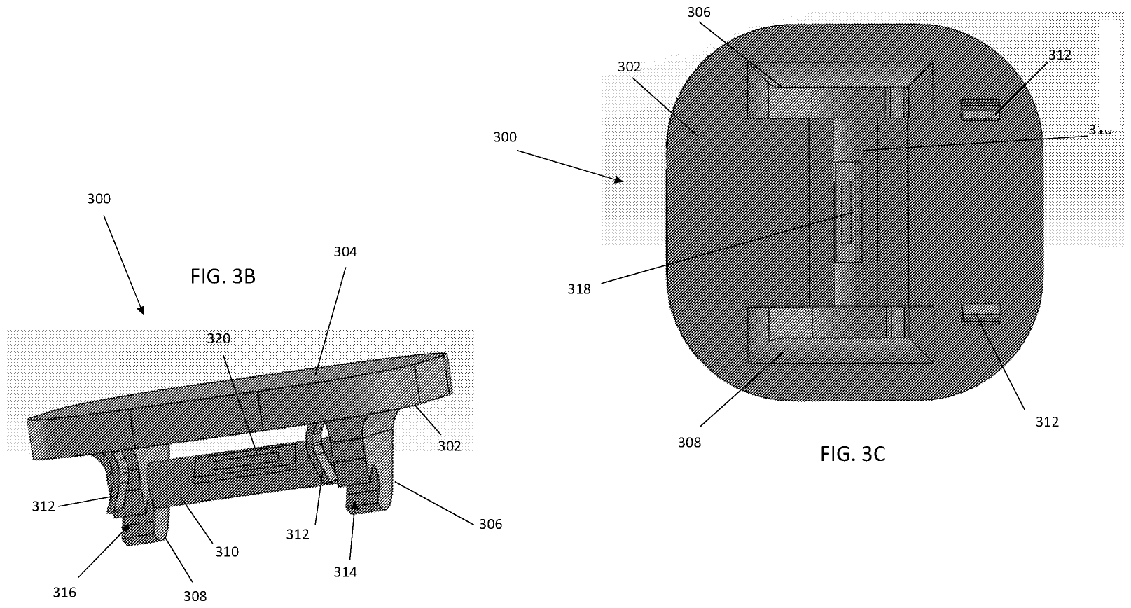

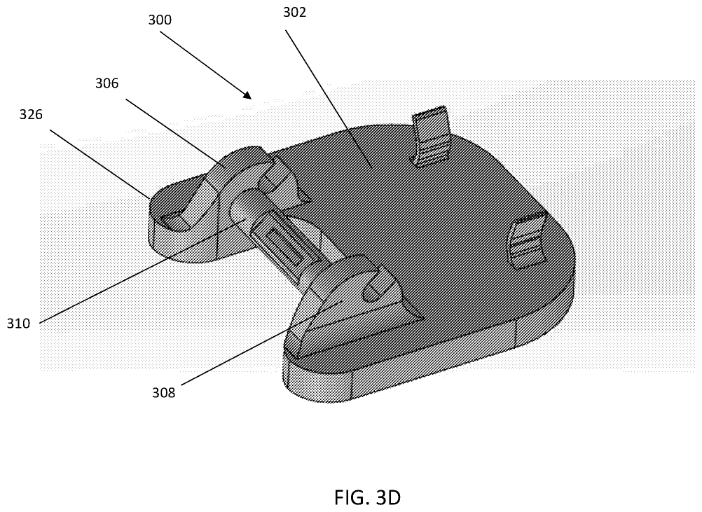

FIGS. 3A-3C depict perspective views of the base plate 300 of the fluid applicator device 100 of FIGS. 1A-1B. The base plate 300 is configured to lock in and couple with the handle 200 (referring to FIGS. 1A-1B) and to provide a base for the foam applicator 400 (referring to FIGS. 2A-2E). The base plate 300 can have any suitable size and shape. In accordance with these embodiments, the base plate 300 can be made out of a plurality of suitable materials. For instance, the base plate 300 can be made out of metals, nonmetals, polymers, composites, ceramics, and any combination thereof. For example, the base plate 300 can be made of polypropylene. The base plate 300 can further be manufactured in a plurality of ways, including injection molding

As illustrated, the base plate 300 has a top surface 302, a bottom surface 304, a first support 306, a second support 308, and a rod 310. In embodiments, the foam applicator 400 can be coupled to the bottom surface 304 opposite from the top surface 302. The foam applicator 400 can be coupled to the bottom surface 3004 by any known methods, such as but not limited to, adhesive attachment, ultrasonic and heat welding, and mechanical entanglement. The base plate 300 can further comprise a set of flexible fins 312 extending from the top surface 302 configured to hold the handle adjacent the base plate 300 while in the delivery condition. The fins 312 are flexible and can bias toward and away from each other to accommodate the handle 200 moving between the delivery and use conditions. The fins 312 can be made of the same material as the base plate 300 or can be made of a different material. As shown, the first support 306 and the second support 308 can be disposed on the top surface 302. The first support 306 can comprise a first slot 314 defined therein, and the second support 308 can comprise a second slot 316 defined therein. Each of the first slot 314 and the second slot 316 can have any suitable size and shape. As illustrated, each of the first slot 314 and the second slot 316 is disposed at an angle with respect to the top surface 302. Without limitations, the angle can be selected from a range up to about 90° in relation to the top surface 302 of the base plate 300. The first slot 314 and the second slot 316 can be configured to receive a portion of the second end 204 (referring to FIGS. 1A-1B) of the handle 200 when coupled to the base plate 300.

The rod 310 can be disposed between the first support 306 and the second support 308. The rod 310 can have any suitable size and shape and can be manufactured with the base as an injection molded component. While illustrated in a cylindrical shape, the rode 310 is not limited to such a shape. In accordance with these embodiments, the rod 310 can be made out of a plurality of suitable materials. For instance, the rod 310 can be made out of metals, nonmetals, polymers, composites, ceramics, and any combination thereof. For example, the rod 310 may comprise polypropylene. As illustrated, the rod 310 can comprise a first indention 318 and a second indention 320. In the present embodiment, the first indention 318 and the second indention 320 can be disposed about a central location along the rod 310. The first indention 318 can be disposed opposite to the second indention 320 to face an upward direction, wherein the second indention 320 can face a downward direction. Each of the first indention 318 and the second indention 320 can have any suitable size and shape. Similar to the first and second slots 314, 316, the first indention 318 and the second indention 320 can be configured to receive a portion of the second end 204 of the handle 200 when coupled to the base plate 300.

FIG. 3D depicts a perspective view of another embodiment of the base plate 300 suitable for the fluid applicator device 100 of FIGS. 1A-1B. Similar to the base plate 300 illustrated in FIGS. 3A-3C, the base plate 300 of FIG. 3D is configured to lock in and couple with the handle 200 (referring to FIGS. 1A-1B) and to provide a base for the foam applicator 400 (referring to FIGS. 2A-2E). In the embodiment of FIGS. 3A-3C, the first support 306, the second support 308, and the rod 310 were disposed at about a central or middle location of the top surface 302 of the base plate 300. As depicted in FIG. 3D, the first support 306 and second support 308 can be disposed at a side 326 of the base plate 300, wherein the rod 310 is disposed between each of the first support 306 and the second support 308. This location of the first support 306, second support 308, and rod 310 can decrease the overall thickness and side profile of the fluid applicator device 100 while in a delivery condition. Although shown along a first side 326, the first support 306 and second support 308 can be disposed along any suitable side of the base plate 300, such as a second side 328 opposite the fins.

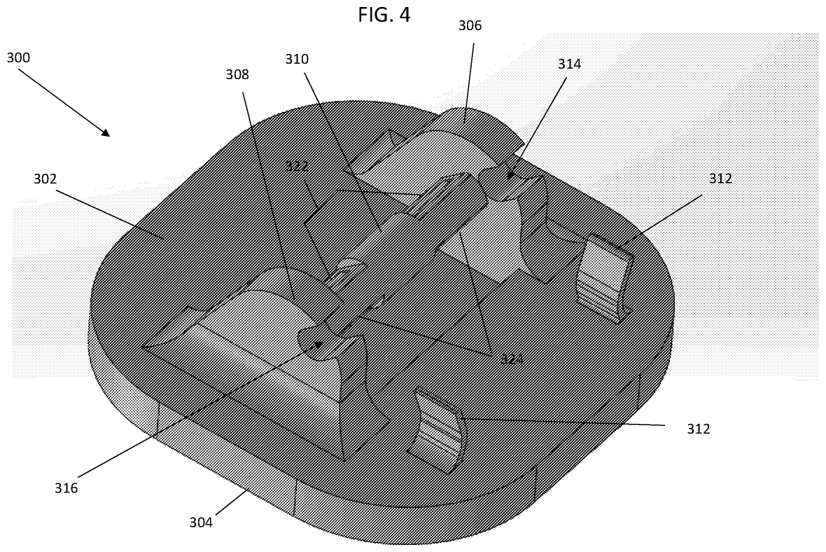

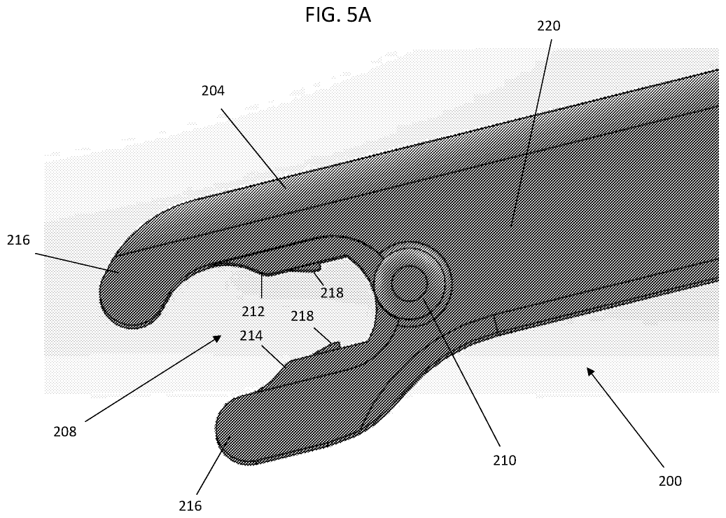

FIG. 4 depicts a perspective view of another embodiment of the base plate 300 of the fluid applicator device 100 of FIGS. 1A-1B. Similar to the base plate 300 illustrated in FIGS. 3A-3C, the current embodiment comprises the top surface 302, bottom surface 304, first support 306, second support 308, rod 310, set of fins 312, first slot 314, and the second slot 316. However, the rod 310 of the current embodiment can include a different configuration of indentions (such as first indention 318 and second indention 320). For example, the rod 310 can comprise a first set of indentions 322 and a second set of indentions 324. The first set of indentions 322 and second set of indentions 324 can operate and function similarly to first indention 318 and second indention 320. The first set of indentions 322 can be disposed opposite to the second set of indentions 324 to face an upward direction, wherein the second set of indentions 324 can face a downward direction. As shown, the first set of indentions 322 and second set of indentions 324 can be disposed at the ends of the rod 310 instead of at a central location of the rod 310. For example, a first one of the first set of indentions 322 can be disposed at one end of rod 310 and a second one of the first set of indentions 322 can be disposed at an opposite end of rod 310. Likewise, a first one of the second set of indentions 324 can be disposed at one end of rod 310 and a second one of the second set of indentions 324 can be disposed at an opposite end of rod 310.

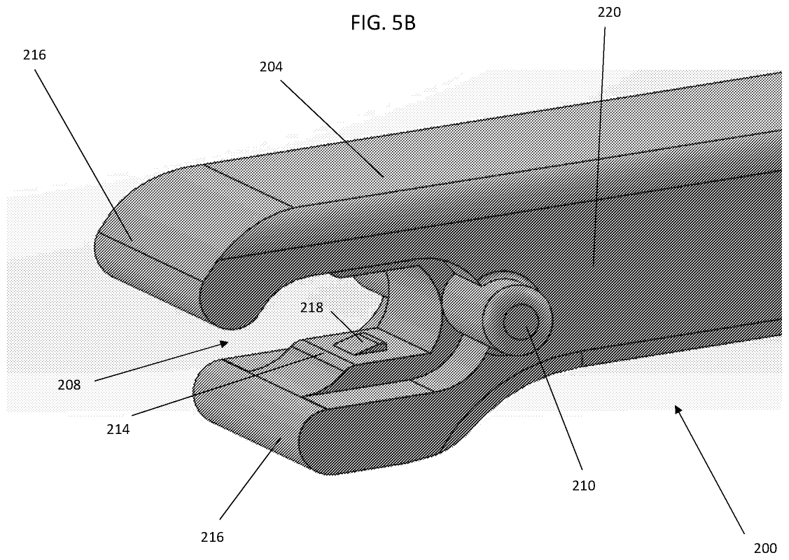

FIGS. 5A-5B depict perspective views of the handle 200 of the fluid applicator device 100 of FIGS. 1A-1B. The handle 200 is configured to at least partially rotate about the rod 310 (referring to FIGS. 3A-4) of the base plate 300 (referring to FIGS. 3A-4) and is configured to lock with the rod 310 in a use condition. As illustrated, the handle 200 has the second end 204, an opening 208, at least one protrusion 210, a first mating member 212, and a second mating member 214. The opening 208 can be defined by two fingers 216 extending from the second end 204 of the handle 200, wherein the opening 208 is between the two fingers 216. Each finger 216 can have any suitable size and shape. For example, the fingers 216 can comprise curvilinear surfaces so as to curve inwards as they extend from the second end 204. As shown, the first mating member 212 and the second mating member 214 can be disposed within the opening 208 between the extending fingers 216. The first mating member 212 can be receivable by the first indention 318 (referring to FIGS. 3A-3C) of the rod 310 in order to lock the handle 200 with the base plate 300. Similarly, the second mating member 214 can be receivable by the second indention 320 (referring to FIGS. 3A-3C) of the rod 310. Each one of the first mating member 212 and the second mating member 214 can comprise a cammed surface 218 that complements the first indentation 318 and/or second indention 320 to prevent the handle 200 from unlocking with the base plate 300 once the device is in the use condition.

The at least one protrusion 210 of the handle 200 can extend from a first side 220 of the handle 200 in order to lock the handle 200 at the desired predetermined angle with the base plate 300. In one or more embodiments, the at least one protrusion 210 can be received by the first slot 314 (referring to FIGS. 3A-4) of the first support 306 (referring to FIGS. 3A-4). The at least one protrusion 210 can be received by the first slot 314 as the first mating member 212 is received by the first indention 318. In embodiments, there can be two protrusions 210. A second protrusion 210 can extend from the handle along a side opposite to the first side 220 and can be received by the second slot 316 (referring to FIGS. 3A-4) of the second support 308 (referring to FIGS. 3A-4).

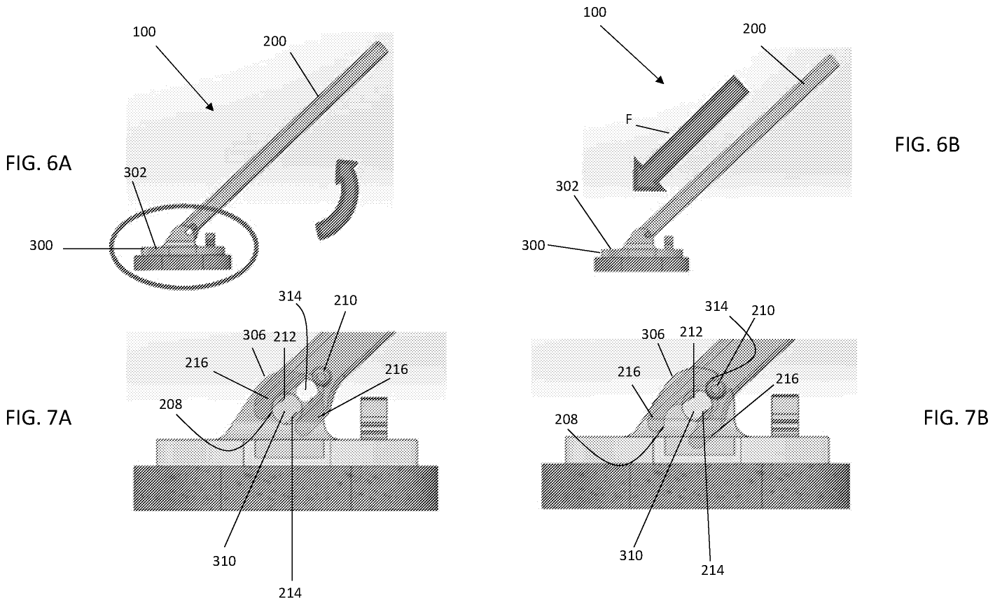

FIGS. 6A-6B depict side views of assembling the fluid applicator device 100 of FIGS. 1A-1B. FIGS. 7A-7B depict cross-sectional views of FIGS. 6A-6B. As embodied herein, the fluid applicator device 100 can initially be in a delivery condition. The delivery condition can be wherein the handle 200 is substantially parallel to the base plate 300, wherein the top surface 302 of the base plate 300 is proximate the handle 200. In the delivery condition, the rod 310 can be at least partially disposed within the opening 208 and between the fingers 216. A user can grip the handle 200 and rotate the handle 200 about the rod 310 to transition to a use condition, as illustrated. The user can apply an axial force F along the handle 200 towards the base plate 300 to lock the handle with the base plate 300 in the use condition. As the axial force is applied, the at least one protrusion 210 can engage with the first support 306 and be received by the first slot 314. As the first slot 314 receives the at least one protrusion 210, the first mating member 212 and the second mating member 214 can engage with the first indentation 318 (referring to FIGS. 3A-3C) and second indention 320 (referring to FIGS. 3A-3C), respectively, to lock the handle 200 to the base plate 300 at the designated angle. In embodiments, the handle 200 snap-fits with the base plate 300.

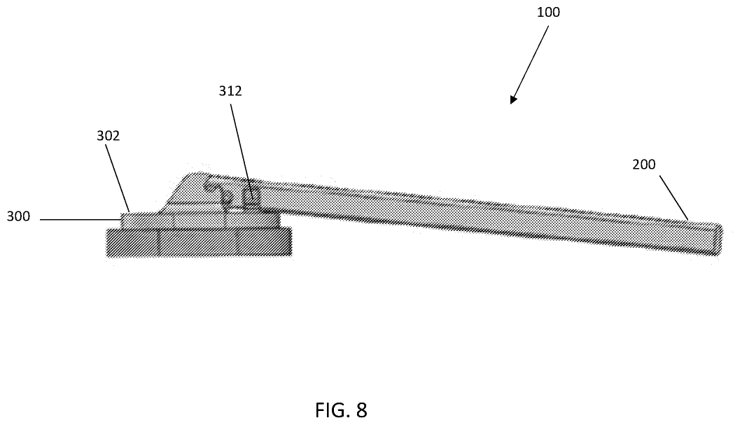

FIG. 8 depicts a side view of the fluid applicator device 100 of FIGS. 1A-1B in a delivery condition. The fluid applicator device 100 can be removed from a packaging (not shown) in the illustrated configuration. The handle 200 is disposed in a direction substantially parallel to the base plate 300, or at a small angle with respect to the base plate 300 (for example, 5 degrees). The handle 200 may abut at least a portion of the top surface 302 of the base plate 300. Further, the handle 200 may be disposed between the set of fins 312 extending from the top surface 302. The set of fins 312 provide support and inhibit lateral movement of the handle 200 with respect to the base plate 300.

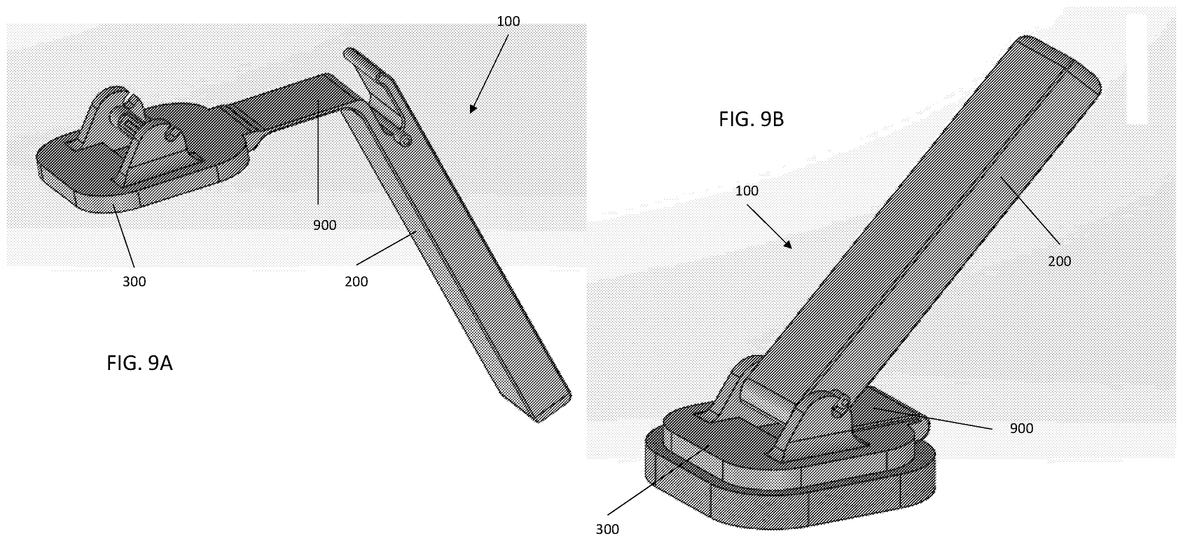

FIGS. 9A-9B depict perspective views of another embodiment of a fluid applicator device with a living hinge 900. As embodied herein, the handle 200 can be coupled to the base plate 300 via the living hinge 900, wherein the handle 200 and base plate 300 may functionally be a singular or monolithic component. For example, the living hinge 900 can be a thin, flexible hinge comprising the same material as the two components coupled therethrough (such as the handle 200 and base plate 300). The living hinge 900 can bend in order to secure the opening 208 (referring to FIGS. 5A-5B) of the handle 200 to the rod 310 (referring to FIGS. 3A-3D) of the base plate 300. In accordance with these embodiments, the living hinge 900 can be made out of a plurality of suitable materials. For instance, the living hinge 900 can be made out of metals, nonmetals, polymers, composites, ceramics, and any combination thereof, and can furthermore be manufactured in a plurality of ways including injection molding. In one embodiment for purposes of example, the living hinge 900 is made out of polypropylene.

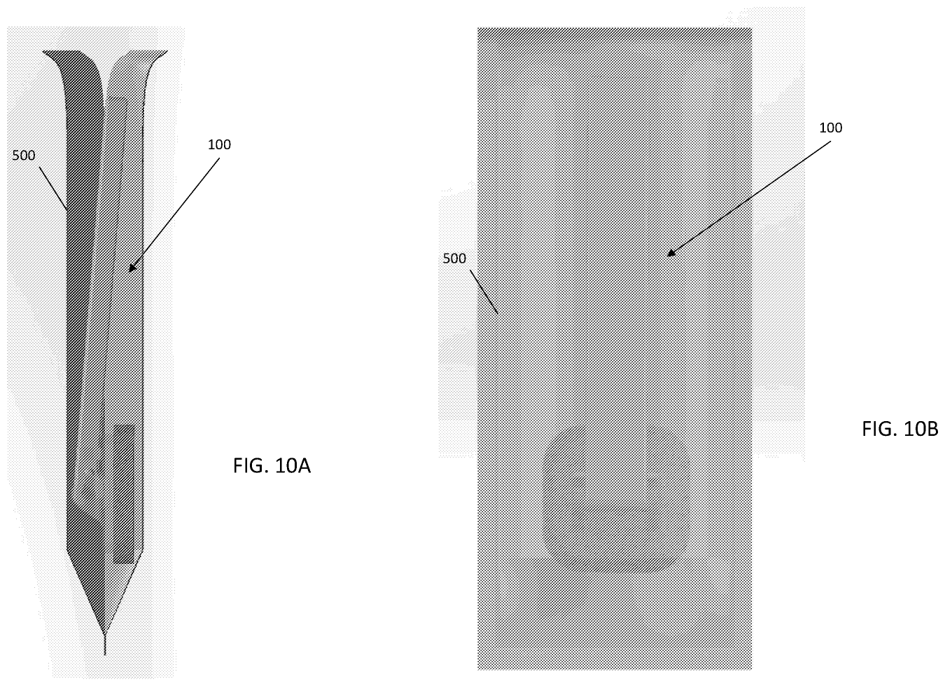

FIGS. 10A-10B depict views of the fluid applicator device 100 of FIGS. 1A-1B disposed in a packaging container 500. FIG. 10A depicts a side view of the fluid applicator device 100 disposed within the packaging container 500, and FIG. 10B depicts a top, plan view of the fluid applicator device 100 disposed within the packaging container 500. A user can transition the fluid applicator device 100 from the delivery condition to the use condition once removed from the packaging container 500 or once the packaging container 500 is at least partially opened. The packaging container 500 can have any suitable size and shape operable to contain the fluid applicator device 100. In accordance with these embodiments, the packaging container 500 can be made out of a plurality of suitable materials. For instance, the packaging container 500 can be made out of metals, nonmetals, polymers, composites, ceramics, and any combination thereof. In embodiments, the packaging container 500 can be made from any suitable material such as but not limited to a relatively flexible resin material, coated papers, shrink wrap, laminate structures, plastic and metalized films and combinations of thereof, calcium carbonate papers or other synthetic paper including various thermoforming packaging solutions. In other embodiments, the packaging containers 500 as shown and described in U.S. Pat. No. 11,160,962 and U.S. Provisional Application No. 63/318,537 can be used with the device according to this disclosure, the contents of each of which is incorporated by reference in their entireties.

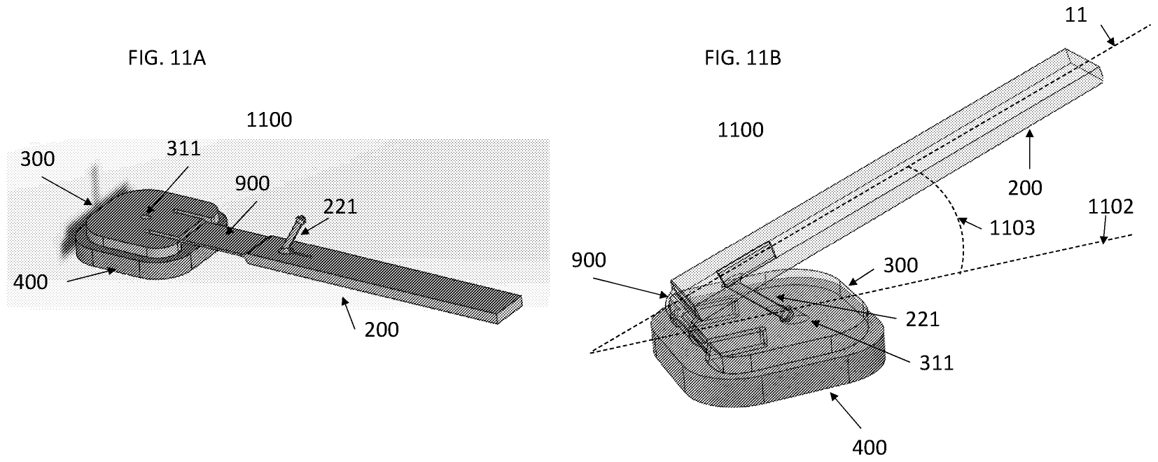

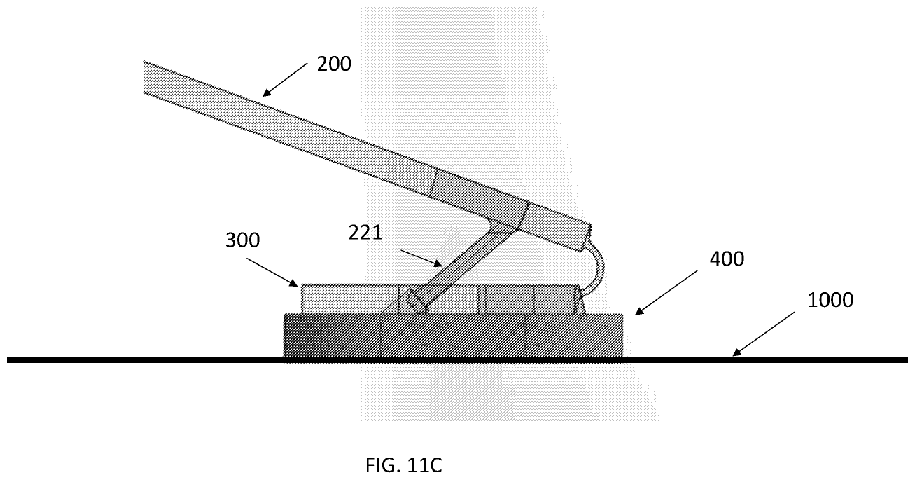

FIGS. 11A-11C depict perspective views and a side view of another embodiment of a fluid applicator device with a living hinge 900. As embodied herein, the handle 200 can be coupled to the base plate 300 via the living hinge 900, wherein the handle 200 and base plate 300 may functionally be a singular or monolithic component. For example, the living hinge 900 can be a thin, flexible hinge comprising the same material as the two components coupled therethrough (such as the handle 200 and base plate 300). The living hinge 900 can bend in order to secure the peg 221 of the handle 200 to the aperture 311 of the base plate 300. Like the embodiments above, the living hinge 900 can be made out of a plurality of suitable materials such as metals, nonmetals, polymers, composites, ceramics, and any combination thereof, and can furthermore be manufactured in a plurality of ways including injection molding. In one embodiment for purposes of example, the living hinge 900 is made out of polypropylene. The foam applicator 400 can be coupled to the bottom of the base plate 300 as described in embodiments above. The mating of the peg 221 and aperture 311 can be achieved mechanically through known methods in the art, including a one way snap fit-locking mechanism such that the connection cannot be undone.

In the use condition, the peg 221 of the handle physically couples with the aperture 311 of the base plate to maintain a relative angle 1103 between the base plate 300 and the handle 200. The angle in the use condition can be predetermined and can range up to about 90°, and more particularly the handle 200 can be disposed at an angle from about 30° to about 60° as maintained by the peg 221. FIG. 11C illustrates a partial phantom side view of the fluid applicator device of FIG. 11B with the peg 221 locked with the base plate 300. The base plate 300 defining the aperture 311 can include a radially outward protrusion within a channel of the aperture, such as a bead or ridge, to lock the peg 221 with the base plate 300 in the use condition. In the use condition, the foam applicator 400 of the fluid applicator device can clean or abrade a surface 1000 to be cleaned, such as a portion of a body to kill microorganisms.

While the disclosed subject matter is described herein in terms of certain embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the system and unit of the disclosed subject matter without departing from the spirit or scope of the disclosed subject matter. Thus, it is intended that the disclosed subject matter include modifications and variations that are within the scope of the appended claims and their equivalents.

What is claimed is:

1. A fluid applicator device, comprising:
a handle having a first end, a second end, and a length therebetween, wherein two fingers extend from the second end and define an opening between the two fingers;
a base plate rotatably coupled with the handle, the base plate having a top surface and a bottom surface, wherein the top surface includes:
a first support and a second support disposed thereon, and
a rod disposed between the first support and the second support, wherein the rod is housed within the opening between the two fingers, the second end of the handle is configured to at least partially rotate about the rod and is configured to lock with the rod in a use condition; and
a foam applicator coupled to the bottom surface of the base plate configured to absorb and distribute an antiseptic solution, wherein the top surface of the base plate is proximate the handle in a delivery condition and the top surface is disposed at an angle to the handle in the use condition.

2. The fluid applicator device of claim 1, wherein the handle further comprises at least a first protrusion at the second end, wherein the at least first protrusion extends from a first side of the handle to lock the handle at the angle with the base plate.

3. The fluid applicator device of claim 2, wherein the first support comprises a first slot disposed at the angle and configured to receive the first protrusion therein to lock the handle at the angle with the base plate.

4. The fluid applicator device of claim 3, wherein the angle is predetermined and can range up to 90° in relation to the top surface of the base plate.

5. The fluid applicator device of claim 3, wherein the handle is axially translatable along the angle towards the base plate to lock the handle with the base plate in the use condition.

6. The fluid applicator device of claim 5, wherein the rod comprises a first indentation and the handle further comprises a first mating member disposed within the opening between the two fingers, the first mating member is receivable in the first indentation of the rod to lock the handle with the base plate.

7. The fluid applicator device of claim 6, wherein the first mating member includes a cammed surface that complements the first indentation to prevent the handle from unlocking with the base plate once the device is in the use condition.

8. The fluid applicator device of claim 7, wherein the rod further comprises a second indentation and the handle further comprises a second mating member disposed within the opening between the two fingers, the second mating member is receivable in the second indentation to lock the handle with the base plate.

9. The fluid applicator device of claim 8, wherein the second mating member includes a cammed surface that complements the second indentation to prevent the handle from unlocking with the base plate once the device is in the use condition.

10. The fluid applicator device of claim 1, further comprising a set of fins extending from the top surface configured to hold the handle in the delivery condition.

11. The fluid applicator device of claim 1, wherein the handle further comprises grip ribbing at the first end configured to support a hand of the user.

12. The fluid applicator device of claim 1, wherein the handle is coupled to the base plate with at least one of a snap-fit connection or a living hinge.

13. A method of using a fluid applicator, comprising:
providing a fluid applicator device including:
a handle having a first end, a second end, and a length therebetween, wherein two fingers extend from the second end and define an opening between the two fingers;
a base plate rotatably coupled with the handle, the base plate having a top surface and a bottom surface, wherein the top surface includes:
a first support and a second support disposed thereon, and
a rod disposed between the first support and the second support, wherein the rod is housed within the opening between the two fingers, the second end of the handle is configured to at least partially rotate about the rod and is configured to lock with the rod in a use condition; and
a foam applicator coupled to the bottom surface of the base plate configured to absorb and distribute an antiseptic solution, wherein the top surface of the base plate is proximate the handle in a delivery condition and the top surface is disposed at an angle to the handle in the use condition;
rotating the handle from a delivery condition towards a use condition about the rod; and
applying an axial force along the handle towards the base plate to lock the handle with the base plate in the use condition.

14. The method of claim 13, further comprising:
engaging at least a first protrusion extending from a first side of the handle with the first support to lock the handle at the angle with the base plate.

15. The method of claim 13, wherein the angle is predetermined and can range up to about 90° in relation to the top surface of the base plate.

16. The method of claim 13, wherein the rod comprises a first indentation and the handle further comprises a first mating member disposed within the opening between the two fingers.

17. The method of claim 16, further comprising:
engaging the first mating member in the first indentation to lock the handle with the base plate, wherein the first mating member includes a cammed surface that complements the first indentation to prevent the handle from unlocking with the base plate once the device is in the use condition.

18. The method of claim 13, further comprising coupling the handle with the base with at least one of a snap-fitting the rod into the opening of the handle or connecting the handle with the base via living hinge.

19. A fluid applicator device, comprising:
a handle having a first end, a second end, and a length therebetween, wherein two fingers extend from the second end and define an opening between the two fingers, wherein the handle further includes at least a first protrusion at the second end;
a base plate rotatably coupled with the handle, wherein the at least first protrusion of the handle extends from a first side of the handle to lock the handle at an angle with the base plate, the base plate having a top surface and a bottom surface, wherein the angle is predetermined and can range up to about 90° in relation to the top surface of the base plate, wherein the top surface includes:
a first support and a second support disposed thereon, and
a rod disposed between the first support and the second support, wherein the rod is housed within the opening between the two fingers, the second end of the handle is configured to at least partially rotate about the rod and is configured to lock with the rod in a use condition, wherein the rod comprises a first indentation and the handle further comprises a first mating member disposed within the opening between the two fingers, the first mating member is receivable in the first indentation to lock the handle with the base plate; and
a foam applicator coupled to the bottom surface of the base plate configured to absorb and distribute an antiseptic solution, wherein the top surface of the base plate is proximate the handle in a delivery condition and the top surface is disposed at an angle to the handle in the use condition.

20. The fluid applicator device of claim 19, wherein the handle further comprises grip ribbing around the first end configured to support a hand of the user, and wherein the handle is coupled to the base plate using at least one of a snap-fit connection or a living hinge.

21. A fluid applicator device, comprising:
a handle having a first end, a second end, and a length therebetween, the handle further comprising a peg disposed between the first end and the second end;
a base plate rotatably coupled with the handle by a living hinge, the base plate having a top surface and a bottom surface, the base plate further defining an aperture configured to mate with the peg; and
a foam applicator coupled to the bottom surface of the base plate configured to absorb and distribute an antiseptic solution;
wherein the peg is configured to lock the handle with the base plate at an angle in a delivery condition.

22. The fluid applicator device of claim 21, wherein the angle is predetermined and can range from about 30° to about 60° in relation to the top surface of the base plate.
